(19) United States
(12) Patent Application Publication
(10) Pub. No.: US 2016/0058722 A1
(43) Pub. Date: Mar. 3, 2016

(54) METHOD FOR IMPROVING THE OXYGEN-RELEASING ABILITY OF HEMOGLOBIN TO ORGANS AND PERIPHERAL TISSUES IN HUMAN BODIES

(71) Applicant: NATIONAL SUN YAT-SEN UNIVERSITY, Kaohsiung (TW)

(72) Inventors: Chia-Chen WANG, Kaohsiung (TW); Muhammad ZULFAJRI, Kaohsiung (TW); You-Qing YU, Kaohsiung (TW)

(21) Appl. No.: 14/840,548

(22) Filed: Aug. 31, 2015

(30) Foreign Application Priority Data
Sep. 1, 2014 (TW) 103130168

(51) Int. Cl. A61K 31/192

(52) U.S. Cl. CPC A61K 31/192

(57) ABSTRACT

A method for improving the oxygen-releasing ability of hemoglobin to organs and peripheral tissues in human bodies is disclosed by administering ferulic acid to a subject in need thereof to improve the oxygen-releasing ability of hemoglobin to the organs and the peripheral tissues in human bodies. Ferulic acid forms a hydrogen bond with αVal1 of hemoglobin, stabilizing the α1/α2 interface of hemoglobin, further stabilizing the oxygenated hemoglobin, hemoglobin variants, recombinant hemoglobin and hemoglobin-based blood substitutes in the low oxygen affinity “T” state and facilitating the oxygen release to the organs and the peripheral tissues.

### CROSS REFERENCE TO RELATED APPLICATIONS

The application claims the benefit of Taiwan application serial No. 103130168, filed Sep. 1, 2014, the subject matter of which is incorporated herein by reference.

### BACKGROUND OF THE INVENTION

1. Field of the Invention

The present invention generally relates to a method and, more particularly, to a method for improving the oxygen-releasing ability of hemoglobin (Hb) to organs and peripheral tissues in human bodies.

2. Description of the Related Art

Hemoglobin is the oxygen-transport protein in the red blood cells. Hemoglobin in the blood carries oxygen from the respiratory organs (i.e. respiratory tract and lung) to organs and peripheral tissues to provide oxygen to the organs and the peripheral tissues and by doing so to assure the normal physiological functions of the organs and the peripheral tissues.

In normal adult humans, hemoglobin is a hetero-tetramer, consisting of a pair of dissimilar subunits, including α₁, α₂, β₁ and β₂ subunits. While the backbone amino acid sequence determines the primary structure of each subunit, the intra-subunit hydrogen bonds and salt bridges formed within each of the subunits govern the secondary and tertiary structure of the subunits. Moreover, the inter-subunit hydrogen bonds and salt bridges formed between different subunits determine and regulate the quaternary structure of the tetrameric hemoglobin.

The quaternary structure of hemoglobin may exist in two allosteric conformation states, including a high oxygen affinity relaxed state (“R” state) and a low oxygen affinity tensed state (“T” state). Hemoglobin can bind oxygen and transform to the “R” state when transported to lungs where the partial pressure of oxygen PO₂ is high, and release the bound oxygen to the organs and the peripheral tissues where the partial pressure of oxygen PO₂ is low, and transform to the “T” state. A number of heterotropic effectors such as pH value, CO₂ and 2,3-bisphosphoglycerate (2,3-BPG) play important roles in regulating the allosteric property of hemoglobin. Moreover, there are six inter-subunit hydrogen bonds in hemoglobin being capable of stabilizing hemoglobin in the low oxygen affinity “T” state, including α₁Arg141---α₂Asp126, α₁Arg141---α₂Lys127, α₁Asp126---α₂Arg141, α₁Lys127---α₂Arg141, β₁His146---α₂Lys40 and β₂His146---α₁Lys40. Among these six “T” state stabilizing inter-subunit contacts, four are related to αArg141 of hemoglobin, pointing to the crucial importance of this residue in sustaining the “T” state.

In general, hemoglobin with an impaired ability of carrying or releasing oxygen may cause a variety of syndromes such as anemia and dizziness; fatigue, weakness and shortness of breath are also frequently found in patients whose hemoglobin has defect oxygen-releasing capability. Syndromes, such as migraine, menstrual disorder and dysmenorrhea are also related with impaired oxygen-delivery efficiency of hemoglobin. Furthermore, insufficient oxygen uptake results in metabolism abnormality and dysfunction of the organs and the peripheral tissues, from which various diseases can begin to develop, including, but not limited to, hypertensions, cardiovascular and neurodegenerative diseases, and growth of carcinogenic cells. The conventional method broadly adopted to treat anemia involves transfusion of normal functional blood. However, this is a passive way of treatment and additional treatments must always be accompanied to alleviate the accompanying adverse side effects. For example, the iron-chelating agent must be applied to patients receiving the blood transfusion in order to down-regulate the iron level in blood to prevent iron-poisoning. In light of this, it is necessary to develop new strategies to improve the oxygen-releasing ability of hemoglobin to the organs and the peripheral tissues in human bodies and to treat various syndromes and diseases related with deficient oxygen delivery.

### SUMMARY OF THE INVENTION

It is therefore the objective of this invention to provide a method for improving the oxygen-releasing ability of hemoglobin to organs and peripheral tissues in human bodies, with ferulic acid used as the active ingredient for improving the oxygen-releasing ability of hemoglobin to the organs and the peripheral tissues in human bodies.

One embodiment of the invention discloses a method for improving the oxygen-releasing ability of hemoglobin to organs and peripheral tissues in human bodies, by administering ferulic acid to a subject in need thereof to improve the oxygen-releasing ability of hemoglobin to the organs and the peripheral tissues in human bodies, wherein ferulic acid forms a hydrogen bond with αVal1 of hemoglobin, stabilizing the key inter-subunit hydrogen bond between α₁Arg141 and α₂Lys127 (and/or symmetrically, between α₁Lys127 and α₂Arg141) of hemoglobin at the α1/α2 interface of hemoglobin by facilitating the formation of the intra-subunit hydrogen bond between α₁Val1 and α₁Lys127 (and/or symmetrically, between α₂Val1 and α₂Lys127) of hemoglobin, thus stabilizing the oxygenated hemoglobin in the low oxygen affinity “T” state and by doing so facilitating the oxygen release to the organs and the peripheral tissues.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 is a diagram illustrating the chemical structure of ferulic acid of the invention.

FIG. 2a is a diagram illustrating the suppression of the “R” state for the oxygenated hemoglobin treated with varying mole ratios of 2,3-BPG to hemoglobin tetramer.

FIG. 2b is a diagram illustrating the suppression of the “R” state for the oxygenated hemoglobin treated with varying mole ratios of ferulic acid of the invention to hemoglobin tetramer.

In the various figures of the drawings, the same numerals designate the same or similar parts. Furthermore, when the term “first”, “second”, “third”, “fourth”, “inner”, “outer”, “top”, “bottom” and similar terms are used hereinafter, it should be understood that these terms refer only to the structure shown in the drawings as it would appear to a person viewing the drawings, and are utilized only to facilitate describing the invention.

### DETAILED DESCRIPTION OF THE INVENTION

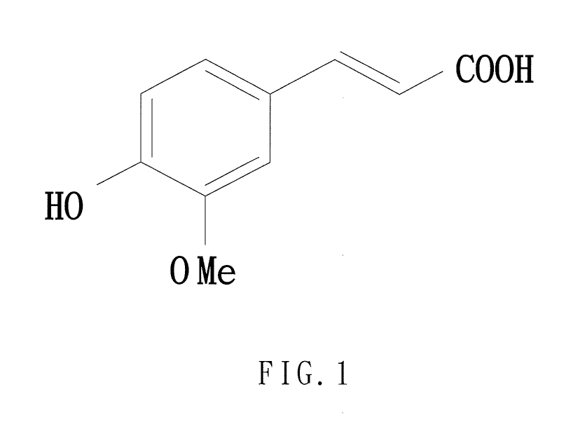

Ferulic acid (shown in FIG. 1) according to the preferred teachings of the invention is able to form at least one hydrogen bond with αVal1 of hemoglobin, stabilizing the key inter-subunit hydrogen bond between α₁Arg141 and α₂Lys127 (and/or symmetrically, between α₁Lys127 and α₂Arg141) of hemoglobin at the α1/α2 interface of hemoglobin by forming the intra-subunit hydrogen bond between α₁Val1 and α₁Lys127 (and/or symmetrically, between α₂Val1 and α₂Lys127) of hemoglobin, further stabilizing the oxygenated hemoglobin in the low oxygen affinity “T” state and facilitating the oxygen release to organs and peripheral tissues. Ferulic acid can be a compound synthesized via organic synthesis approaches. Alternatively, ferulic acid can also be a natural compound extracted from herbs, such as, but not limited to, Angelica sinensis (Oliv.) Diels or Ligusticum chuanxiong Hort.

In the present invention, ferulic acid of the invention can be given to any target individually or combined with any acceptable excipients, for example drug carriers or other ingredients, and is capable of being further manufactured into any form of medicaments, including, but not limited to, oral administration, intravenous injection, intravenous infusion and nasal inhalation for effective delivery to the targets. For the oral administration, ferulic acid of the invention can be manufactured into the form of pill, capsule, powder, solution and pastil. The dosage of the medicaments depends on the form of medicaments, the bioavailability of the corresponding form of medicaments and the medical conditions of individuals. The suggested dosage of ferulic acid is 50-100 mg/kg body weight per day.

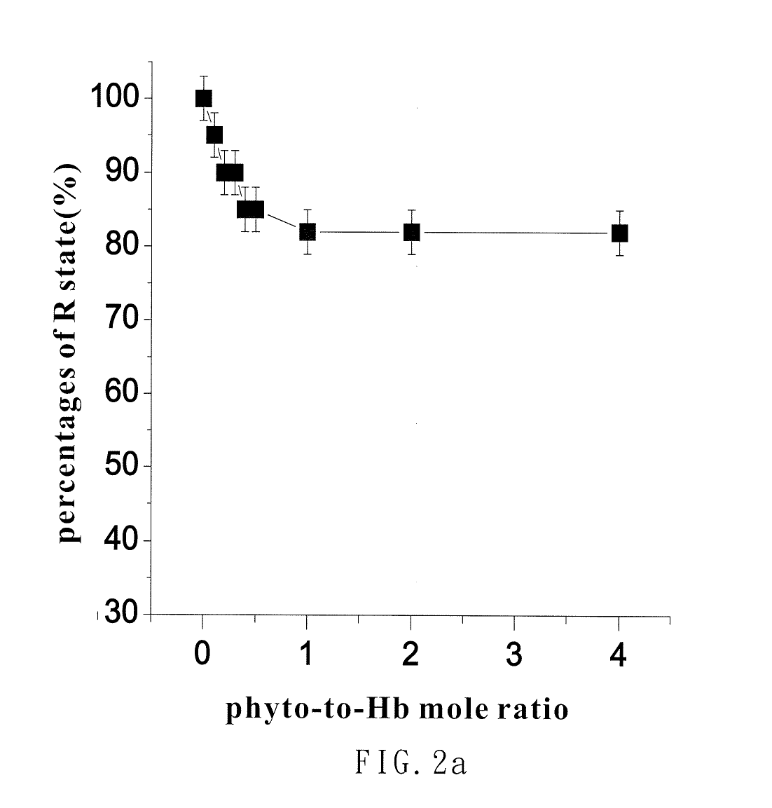

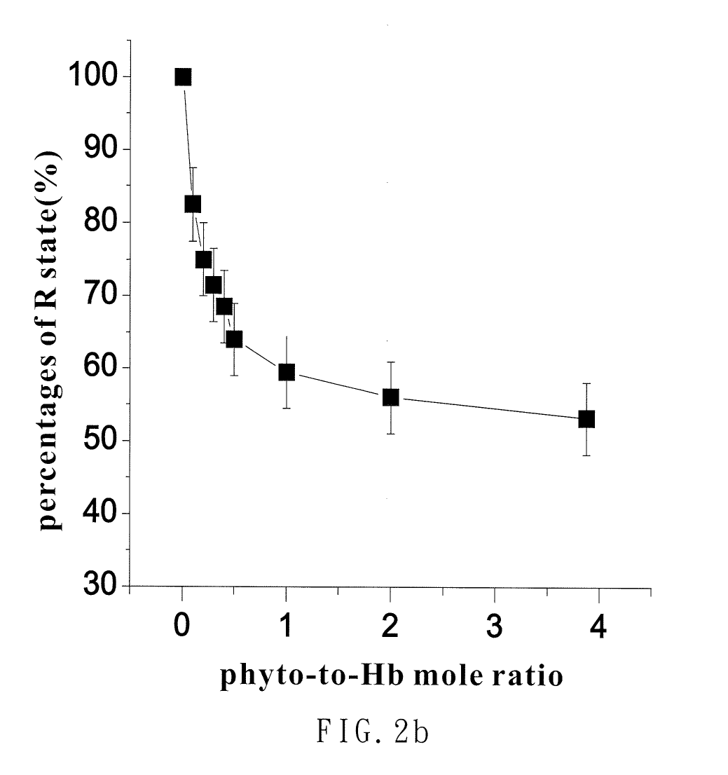

In order to evaluate the effect of ferulic acid on stabilizing the oxygenated hemoglobin in the low oxygen affinity “T” state, compounds including 2,3-BPG (group A0) and ferulic acid (group A1) are mixed with hemoglobin as a function of their mole ratio to hemoglobin, followed by the resonance Raman spectroscopy measurements at 532 nm excitation wavelength under the oxygen atmosphere. The percentages of the high oxygen affinity “R” state for hemoglobin treated with 2,3-BPG (shown in FIG. 2a, as reference) and ferulic acid (shown in FIG. 2b) are analyzed from the resonance Raman spectroscopy measurements. In more specific, the percentage of high oxygen affinity “R” state for each treated hemoglobin was obtained by fitting a simulated spectrum comprised of adjustable weighing factors of the T and R states of pure hemoglobin to the obtained resonance Raman spectrum of treated hemoglobin.

Referring to FIG. 2a, 2,3-BPG of group A0 reduces the relative ratio of the high oxygen affinity “R” state for oxygenated hemoglobin, demonstrating that 2,3-BPG stabilizes the low oxygen affinity “T” state and inhibits the transformation from the low oxygen affinity “T” state to the high oxygen affinity “R” state, with the “R” state suppression efficiency of about 20%. Moreover, referring to FIG. 2b, ferulic acid can also stabilize the low oxygen affinity “T” state and inhibit its transformation from the low oxygen affinity “T” state to the high oxygen affinity “R” state with the “R” state suppression efficiency higher than 50% shown in FIG. 2a (group A0).

In addition, the active sites of oxygenated hemoglobin are analyzed by the computational docking analysis. Ferulic acid forms a hydrogen bond with αVal1 of hemoglobin, facilitating the formation of the intra-subunit hydrogen bond between α₁Val1 and α₁Lys127 (and/or symmetrically, between α₂Val1 and α₂Lys127) of hemoglobin. By doing so, ferulic acid can aid to stabilize the key inter-subunit hydrogen bond between α₁Arg141 and α₂Lys127 (and/or symmetrically, between α₁Lys127 and α₂Arg141) of hemoglobin at the α1/α2 interface of hemoglobin, thus stabilizing the oxygenated hemoglobin in the low oxygen affinity “T” state and facilitating the oxygen release to the organs and the peripheral tissues.

In conclusion, ferulic acid according to the invention can form a hydrogen bond with αVal1 of hemoglobin, stabilizing the oxygenated hemoglobin in the low oxygen affinity “T” state and thus facilitating the oxygen release to the organs and the peripheral tissues. Therefore, ferulic acid according to the invention can be used for improving the oxygen-releasing ability of hemoglobin to the organs and the peripheral tissues in human bodies, and further improving conditions or diseases caused by anoxia, such as anemia, migraine, dysmenorrhea, hypertension and the corresponding diseases.

Moreover, ferulic acid according to the invention can enhance and thus ensure sufficient oxygen uptake, preventing from metabolism abnormality of the organs and the peripheral tissues. Therefore, ferulic acid according to the invention poses the therapeutic effects on preventing from structural and functional abnormalities of tissue cells and related biomolecules or growth of carcinogenic cells, and can be further used for protecting from cardiovascular diseases, neurodegenerative diseases and cancers.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. A method for improving the oxygen-releasing ability of hemoglobin to organs and peripheral tissues in human bodies, by administering ferulic acid to a subject in need thereof to improve the oxygen-releasing ability of hemoglobin to the organs and the peripheral tissues in human bodies, wherein ferulic acid forms a hydrogen bond with αVal1 of hemoglobin, strengthening the α1/α2 interface of hemoglobin, further stabilizing the oxygenated hemoglobin in the low oxygen affinity “T” state and facilitating the oxygen release to the organs and the peripheral tissues.

2. The method for improving the oxygen-releasing ability of hemoglobin to organs and peripheral tissues in human bodies as claimed in claim 1, wherein ferulic acid is administered to the subject in need thereof in a dose of 50-100 mg/per kilogram of body weight per day.
